(19) United States
(12) Patent Application Publication
(10) Pub. No.: US 2016/0045723 A1
(43) Pub. Date: Feb. 18, 2016

(54) IMPLANTABLE NEUROSTIMULATION SYSTEMS AND METHODS THEREOF

(71) Applicant: PACESETTER, INC., Sylmar, CA (US)

(72) Inventors: Gene A. Bornzin, Simi Valley, CA (US); Zoltan Somogyi, Simi Valley, CA (US); Kevin Wilson, McKinney, CA (US)

(21) Appl. No.: 14/462,329

(22) Filed: Aug. 18, 2014

(51) Int. Cl. A61N 1/05; A61N 1/36

(52) U.S. Cl. CPC A61N 1/0534; A61N 1/36125

(57) ABSTRACT

The present disclosure provides neurostimulation methods and system for deep brain stimulation. A neurostimulation apparatus includes a plug configured to be mounted to a burr hole formed in a skull of a subject, a plurality of cable conductors configured to be coupled to the plug, and a lead configured to be coupled to the plurality of cable conductors, the lead comprising at least one electrode configured to apply stimulation to the subject, wherein the plug is configured to generate and supply electrical stimulation pulses to the lead through the plurality of cable conductors.

### FIELD OF THE DISCLOSURE

The present disclosure relates generally to neurostimulation methods, systems, and more particularly to a neurostimulation system for deep brain stimulation including a plug that functions as an internal pulse generator.

### BACKGROUND ART

Neurostimulation is a treatment method utilized for managing the disabilities associated with pain, movement disorders such as Parkinson's Disease (PD), dystonia, and essential tremor, and also a number of psychological disorders such as depression, mood, anxiety, addiction, and obsessive compulsive disorders. Deep brain stimulation systems deliver stimulation to a patient's brain.

At least some known deep brain stimulation systems include a plug that mounts to a burr hole formed in the patient's skull. A lead including stimulation electrodes is coupled to the plug. The plug includes mechanical features (e.g., a clamping mechanism) that lock the lead in place over an exit point from the patient's brain. The lead exits the brain, and is coupled to a stimulation device via an extension. The stimulation device may be located, for example, in the patient's chest.

Accordingly, at least some known neurostimulation systems require connecting a lead implanted in a patient's brain to a stimulation device remotely located in another portion of the patient. These neurostimulation systems may be relatively expensive and uncomfortable for the patient. For example, such neurostimulation systems may require multiple surgeries to implant.

### BRIEF SUMMARY OF THE DISCLOSURE

In one embodiment, the present disclosure is directed to a neurostimulation apparatus for deep brain stimulation. The neurostimulation apparatus includes a plug configured to be mounted to a burr hole formed in a skull of a subject, a plurality of cable conductors configured to be coupled to the plug, and a lead configured to be coupled to the plurality of cable conductors, the lead comprising at least one electrode configured to apply stimulation to the subject, wherein the plug is configured to generate and supply electrical stimulation pulses to the lead through the plurality of cable conductors.

In another embodiment, the present disclosure is directed to a neurostimulation system. The neurostimulation system includes an apparatus for deep brain stimulation that includes a plug configured to be mounted to a burr hole formed in a skull of a subject, a plurality of cable conductors coupled to the plug, and a lead coupled to the plurality of cable conductors, the lead comprising at least one electrode configured to apply stimulation to the subject, wherein the plug is configured to generate and supply electrical stimulation pulses to the lead through the plurality of cable conductors. The neurostimulation system further includes a controller device communicatively coupled to the apparatus and configured to control operation of the apparatus.

In another embodiment, the present disclosure is directed to a method for implanting a neurostimulation apparatus for deep brain stimulation. The method includes inserting a lead into a brain of a subject, the lead including at least one electrode configured to apply stimulation to the subject, coupling a plurality of cable conductors to the lead, coupling the plurality of cable conductors to a plug, and mounting the plug to a burr hole formed in a skull of the subject, wherein the plug is configured to generate and supply electrical stimulation pulses to the lead through the plurality of cable conductors.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic view of one embodiment of a stimulation system.

FIGS. 2A-2C are schematic views of stimulation portions that may be used with stimulation system of FIG. 1.

FIG. 3 is a schematic view of one embodiment of a neurostimulation apparatus.

FIGS. 4A-4C are schematic views of different embodiments of lead assemblies that may be used with the neurostimulation apparatus of FIG. 3.

FIGS. 5A and 5B are schematic views of different embodiments of connections between a lead assembly and a plug.

FIG. 6 is a schematic view of one embodiment of a cannula that may be used to implant the neurostimulation apparatus of FIG. 3.

FIGS. 7A and 7B are schematic views of an embodiment of a lead assembly that includes a split segment of tubing.

FIGS. 8A and 8B are schematic views of an embodiment of a lead assembly that includes cable conductors arranged in a helix configuration.

Corresponding reference characters indicate corresponding parts throughout the several views of the drawings.

### DETAILED DESCRIPTION OF THE DISCLOSURE

The present disclosure provides neurostimulation methods and system for deep brain stimulation. A neurostimulation apparatus includes a plug configured to be mounted to a burr hole formed in a skull of a subject, a plurality of cable conductors configured to be coupled to the plug, and a lead configured to be coupled to the plurality of cable conductors, the lead comprising at least one electrode configured to apply stimulation to the subject, wherein the plug is configured to generate and supply electrical stimulation pulses to the lead through the plurality of cable conductors.

Neurostimulation systems are devices that generate electrical pulses and deliver the pulses to nerve tissue of a patient to treat a variety of disorders. Spinal cord stimulation (SCS) is the most common type of neurostimulation within the broader field of neuromodulation. In SCS, electrical pulses are delivered to nerve tissue in the spine typically for the purpose of chronic pain control. While a precise understanding of the interaction between the applied electrical energy and the nervous tissue is not fully appreciated, it is known that application of an electrical field to spinal nervous tissue can effectively mask certain types of pain transmitted from regions of the body associated with the stimulated nerve tissue. Specifically, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

SCS systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals (also electrically coupled to the wire conductors) that are adapted to receive electrical pulses. The distal end of a respective stimulation lead is implanted within the epidural space to deliver the electrical pulses to the appropriate nerve tissue within the spinal cord that corresponds to the dermatome(s) in which the patient experiences chronic pain. The stimulation leads are then tunneled to another location within the patient's body to be electrically connected with a pulse generator or, alternatively, to an “extension.”

The pulse generator is typically implanted within a subcutaneous pocket created during the implantation procedure. In SCS, the subcutaneous pocket is typically disposed in a lower back region, although subclavicular implantations and lower abdominal implantations are commonly employed for other types of neuromodulation therapies.

The pulse generator is typically implemented using a metallic housing that encloses circuitry for generating the electrical pulses, control circuitry, communication circuitry, a rechargeable battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on a stimulation lead.

Peripheral nerve field stimulation (PNFS) is another form of neuromodulation. The basic devices employed for PNFS are similar to the devices employed for SCS including pulse generators and stimulation leads. In PNFS, the stimulation leads are placed in subcutaneous tissue (hypodermis) in the area in which the patient experiences pain. Electrical stimulation is applied to nerve fibers in the painful area. PNFS has been suggested as a therapy for a variety of conditions such as migraine, occipital neuralgia, trigeminal neuralgia, lower back pain, chronic abdominal pain, chronic pain in the extremities, and other conditions.

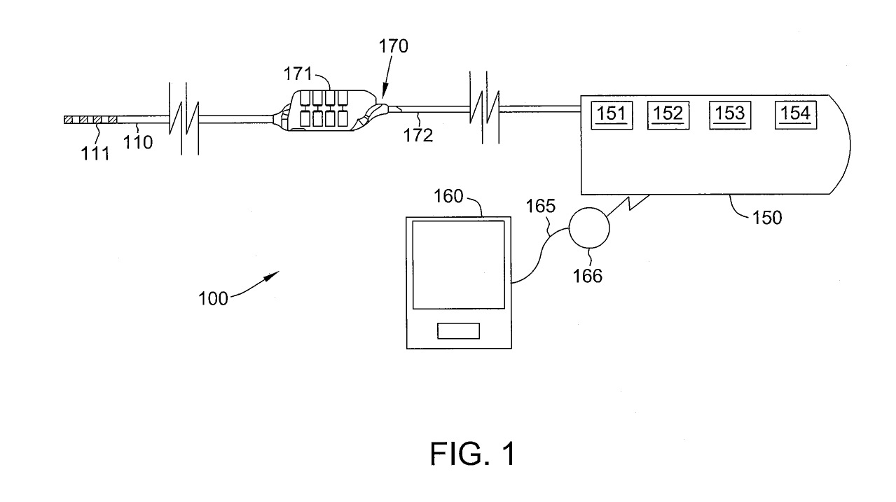

Referring now to the drawings, and in particular to FIG. 1, a stimulation system is indicated generally at 100. Stimulation system 100 generates electrical pulses for application to tissue of a patient, or subject, according to one embodiment. System 100 includes an implantable pulse generator 150 that is adapted to generate electrical pulses for application to tissue of a patient. Implantable pulse generator 150 typically includes a metallic housing that encloses a controller 151, pulse generating circuitry 152, a battery 153, far-field and/or near field communication circuitry 154, and other appropriate circuitry and components of the device. Controller 151 typically includes a microcontroller or other suitable processor for controlling the various other components of the device. Software code is typically stored in memory of pulse generator 150 for execution by the microcontroller or processor to control the various components of the device.

Pulse generator 150 may comprise one or more attached extension components 170 or be connected to one or more separate extension components 170. Alternatively, one or more stimulation leads 110 may be connected directly to pulse generator 150. Within pulse generator 150, electrical pulses are generated by pulse generating circuitry 152 and are provided to switching circuitry. The switching circuit connects to output wires, traces, lines, or the like (not shown) which are, in turn, electrically coupled to internal conductive wires (not shown) of a lead body 172 of extension component 170. The conductive wires, in turn, are electrically coupled to electrical connectors (e.g., “Bal-Seal” connectors) within connector portion 171 of extension component 170. The terminals of one or more stimulation leads 110 are inserted within connector portion 171 for electrical connection with respective connectors. Thereby, the pulses originating from pulse generator 150 and conducted through the conductors of lead body 172 are provided to stimulation lead 110. The pulses are then conducted through the conductors of lead 110 and applied to tissue of a patient via electrodes 111. Any suitable known or later developed design may be employed for connector portion 171.

For implementation of the components within pulse generator 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 20060170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within pulse generator 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number of electrodes 111, terminals, and internal conductors.

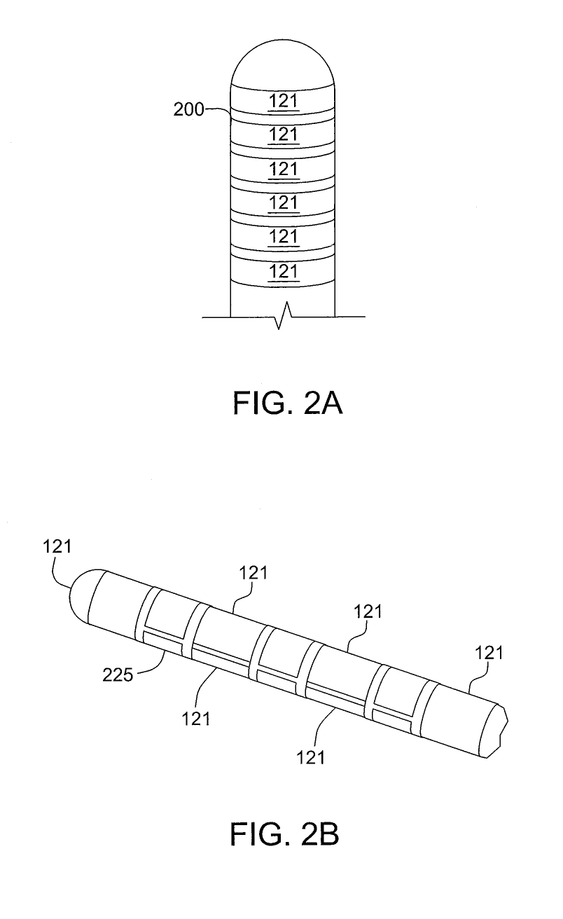

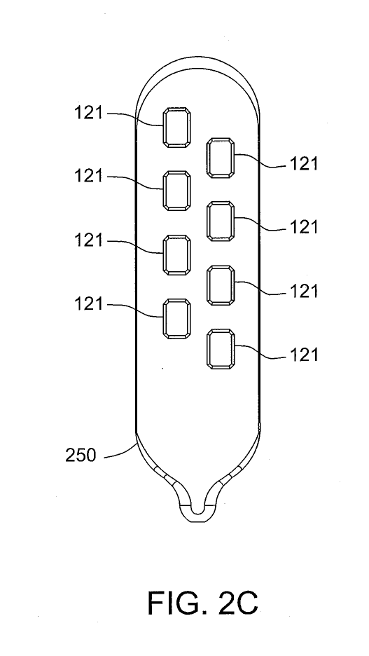

FIGS. 2A-2C respectively depict stimulation portions 200, 225, and 250 for inclusion at the distal end of lead 110. Stimulation portion 200 depicts a conventional stimulation portion of a “percutaneous” lead with multiple ring electrodes. Stimulation portion 225 depicts a stimulation portion including several “segmented electrodes.” The term “segmented electrode” is distinguishable from the term “ring electrode.” As used herein, the term “segmented electrode” refers to an electrode of a group of electrodes that are positioned at the same longitudinal location along the longitudinal axis of a lead and that are angularly positioned about the longitudinal axis so they do not overlap and are electrically isolated from one another. Example fabrication processes are disclosed in U.S. Patent Publication No. 2010072657, entitled, “METHOD OF FABRICATING STIMULATION LEAD FOR APPLYING ELECTRICAL STIMULATION TO TISSUE OF A PATIENT,” which is incorporated herein by reference. Stimulation portion 250 includes multiple planar electrodes on a paddle structure.

Controller device 160 may be implemented to recharge battery 153 of pulse generator 150 (although a separate recharging device could alternatively be employed). A “wand” 165 may be electrically connected to controller device through suitable electrical connectors (not shown). The electrical connectors are electrically connected to coil 166 (the “primary” coil) at the distal end of wand 165 through respective wires (not shown). Typically, coil 166 is connected to the wires through capacitors (not shown). Also, in some embodiments, wand 165 may comprise one or more temperature sensors for use during charging operations.

The patient then places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. Controller 160 generates an AC-signal to drive current through coil 166 of wand 165. Assuming that primary coil 166 and secondary coil are suitably positioned relative to each other, the secondary coil is disposed within the field generated by the current driven through primary coil 166. Current is then induced in secondary coil. The current induced in the coil of the implantable pulse generator is rectified and regulated to recharge battery of generator 150. The charging circuitry may also communicate status messages to controller 160 during charging operations using pulse-loading or any other suitable technique. For example, controller 160 may communicate the coupling status, charging status, charge completion status, etc.

External controller device 160 is also a device that permits the operations of pulse generator 150 to be controlled by user after pulse generator 150 is implanted within a patient, although in alternative embodiments separate devices are employed for charging and programming. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. Also, the wireless communication functionality of controller device 160 can be integrated within the handheld device package or provided as a separate attachable device. The interface functionality of controller device 160 is implemented using suitable software code for interacting with the user and using the wireless communication capabilities to conduct communications with IPG 150.

Controller device 160 preferably provides one or more user interfaces to allow the user to operate pulse generator 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc. IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 01/93953, entitled “NEUROMODULATION THERAPY SYSTEM,” and US Pat. No. 7,228,179, entitled “METHOD AND APPARATUS FOR PROVIDING COMPLEX TISSUE STIMULATION PATTERNS,” which are incorporated herein by reference.

Example commercially available neurostimulation systems include the EON MINI™ pulse generator and RAPID PROGRAMMER™ device from St. Jude Medical, Inc. (Plano, Tex.). Example commercially available stimulation leads include the QUATTRODE™, OCTRODE™, AXXESS™ LAMITRODE™, TRIPOLE™, EXCLAIM™, and PENTA™ stimulation leads from St. Jude Medical, Inc.

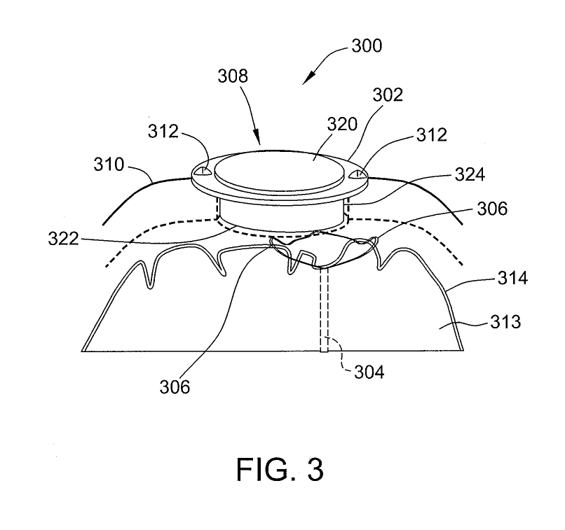

In FIG. 3, an implantable neurostimulation apparatus for deep brain stimulation (DBS) is indicated generally at 300. Apparatus 300 includes a plug 302 coupled to at least one lead 304 via one or more cable conductors 306. As shown in FIG. 3, plug 302 is mounted in a burr hole 308 formed in a skull 310 of the subject. In the illustrated embodiment, plug 302 is mounted to skull 310 using a plurality of fasteners 312 (e.g., screws). Alternatively, plug 302 may be mounted to skull 310 using any techniques that enables apparatus 300 to function as described herein. Apparatus 300 facilitates performing DBS on the subject, as described herein. In some embodiments, a diameter of plug 302 is in a range from approximately 14 millimeters (mm) to 34 mm, and a thickness of plug 302 is in a range from approximately 5 mm to 10 mm. Alternatively, plug 302 may have any suitable dimensions. Further, although plug 302 is illustrated as substantially cylindrical, in other embodiments, other shapes may be utilized.

In this embodiment, plug 302 includes a plurality of components that enable plug 302 to control stimulation applied to the brain 313 of the subject. That is, plug 302 functions as an internal pulse generator, or stimulator, such as IPG 150 (shown in FIG. 1). Accordingly, plug 302 generates and supplies one or more electrical stimulation pulses to stimulating electrodes (not shown in FIG. 3) on lead 304.

To function as a stimulator, plug 302 may include a battery that provides power for plug 302, a charging coil that enables inductively charging plug 302, a connector for connecting plug 302 to cable conductors 306, a communication antennae that facilitates communications between apparatus 300 and an external device (e.g., controller device 160 (shown in FIG. 1)), and processing circuitry for controlling operation of plug 302 and lead 304. The charging coil may collect energy from a wand, a pillow, or a hat with a complementary coil that aligns with plug 302 for inductive charging.

In some embodiments, the processing circuitry is proximate a top 320 of plug 302, the connector is proximate a bottom 322 of plug 302, and the charging coil and communication antennae are proximate a perimeter 324 of plug 302. Alternatively, the components of plug 302 may be arranged in any configuration that enables apparatus 300 to function as described herein.

Because plug 302 functions as an IPG, no wired connections to devices that are outside of skull 310 are required. Accordingly, in contrast to at least some known neurostimulation systems, apparatus 300 is a self-contained, relatively compact device that does not requires additional external connections. As such, neurostimulation apparatus 300 includes less components and may be easier to surgically implant than at least some known neurostimulation systems.

Further, because apparatus 300 does not include any leads that leave skull 310, unlike at least some known neurostimulation systems, apparatus 300 does not require a clamping mechanism or similar device designed to precisely align leads with an exit point out of skull 310. Moreover, because apparatus 300 is relatively compact, unlike neurostimulation systems with additional, longer leads, apparatus 300 may be MRI-compatible. Apparatus 300 may also reduce a risk of infection, as no components exit skull 310.

In the illustrated embodiment, cable conductors 306 are relatively flexible, thread-like conductors. For example, cable conductors 306 may have a diameter between approximately 0.0012 to 0.012 inches, and more particularly, between approximately 0.0025 to 0.006 inches. Because cable conductors 306 are relatively small and flexible, they exert virtually no net force on plug 302 and lead 304. Further, in this embodiment, cable conductors 306 are coated in ethylene tetrafluoroethylene (ETFE) to prevent cable conductors 306 from sticking to the tissue of subject and to provide insulation. As shown in FIG. 3, cable conductors 306 may be arranged on the dura 314 of the subject.

Apparatus 300 includes four cable conductors 306 in this embodiment. Alternatively, because of the small size of cable conductors 306, apparatus 300 may include more (e.g., eight) cable conductors 306. Further, cable conductors 306 may be twisted around one another to form one or more groups (e.g., twisted pairs, twisted trios, twisted quartets, etc.) of cable conductors 306. Because of their flexibility, cable conductors 306 are highly fracture resistant.

The flexibility of cable conductors 306 also provides additional benefits. For example, because cable conductors 306 are flexible, lead 304 can be selectively positioned in a variety of locations relative to plug 302. Accordingly, even if plug 302 is not directly adjacent to a desired stimulation site, lead 304 can still be adjustably positioned at the stimulation site. Further, cable conductors 306 are relatively free to move/migrate over time. Moreover, even if tissue grows over cable conductors 306, due to their flexibility and size, cable conductors 306 can be cut and surgically removed relatively easily.

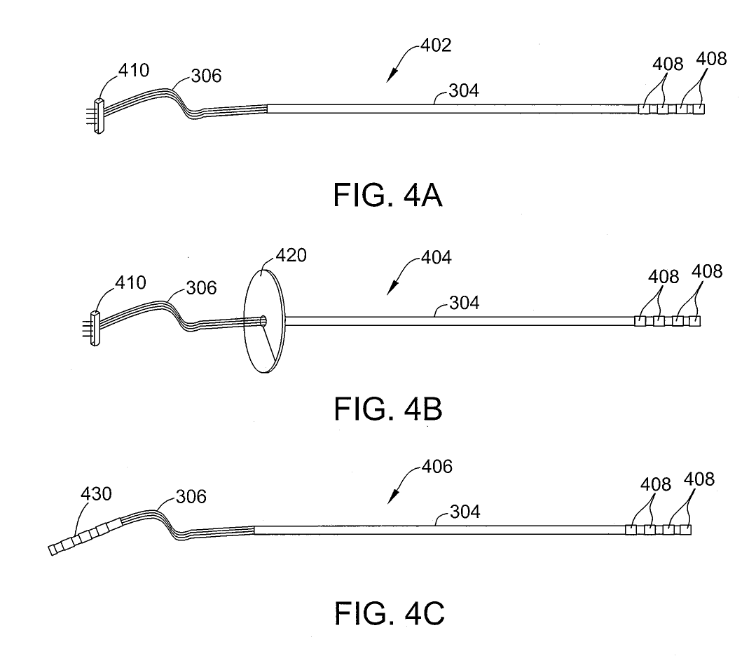

FIGS. 4A-4C are schematic views of different embodiments of lead assemblies 402, 404, and 406. In FIG. 4A, lead assembly 402 includes cable conductors 306 coupled to lead 304. Lead 304 includes a plurality of electrodes 408 for applying stimulation to the subject. In the exemplary embodiment, lead 304 includes four electrodes 408. Alternatively, lead 304 may include any number of electrodes 408 that enables lead assembly 402 to function as described herein. Electrodes 408 may be used for stimulation and/or monitoring purposes. That is, electrodes 408 may apply stimulation to the subject, or may monitor neurological activity (e.g., by measuring local field potentials) of the subject.

For example, in some embodiments, sensing electrodes of electrodes 408 are utilized to measure a trimmer frequency of apparatus 300. A frequency component of the trimmer may be determined, for example, by performing a fast Fourier transform/periodogram averaging for a plurality of 1 second measurement intervals. Once the frequency component is determined, stimulation electrodes may be selected from electrodes 408 to minimize the frequency component.

As shown in FIG. 4A, cable conductors 306 extend between lead 304 and a connector 410. Connector 410 facilitates communicatively coupling cable conductors 306 to plug 302. For example, connector 410 may mate with a complementary connector formed on bottom 322 of plug 302. In the embodiment shown in FIG. 4A, connector 410 is an in-line connector. Using in-line connectors 410, as compared to barrel connectors, reduces an overall size/profile of lead assembly 402.

FIG. 4B is a schematic diagram of an alternative lead assembly 404. Unless otherwise indicated, lead assembly 404 is substantially similar to lead assembly 402 (shown in FIG. 4A). In the embodiment shown in FIG. 4B, a split washer 420 substantially circumscribes cable conductors 306. When implanted in the subject, split washer 420 provides a platform atop dura 314 for cable conductors 306 to rest on. Split washer 420 may also simplify extraction of lead assembly 404, as split washer 420 collects cable conductors 306 in a single location. Split washer 420 may be fabricated from, for example, polyurethane and/or silicone.

In some embodiments, instead of split washer 420, cable conductors 306 are placed on a relatively thin, flexible doily-type structure (not shown) that rests on dura 314. Further, in some embodiments, a cavity for storing excess stretches of cable conductors 306 is formed between two doily-type structures. Storing excess stretches of cable conductors 306 using this technique may reduce magnetic interference at plug 302. The doily-type structures may be fabricated from, for example, silicone. Over time, tissue may grow over the doily-type structure. However, due to its flexibility and thinness, the doily-type structure can be cut and excised relatively easily.

FIG. 4C is a schematic diagram of an alternative lead assembly 406. Unless otherwise indicated, lead assembly 406 is substantially similar to lead assembly 402 (shown in FIG. 4A). In the embodiment shown in FIG. 4C, lead assembly 406 includes a cylindrical barrel connector 430. As with in-line connectors 410 (shown in FIGS. 4A and 4B), cylindrical barrel connector 430 couples to a complementary connector on plug 302.

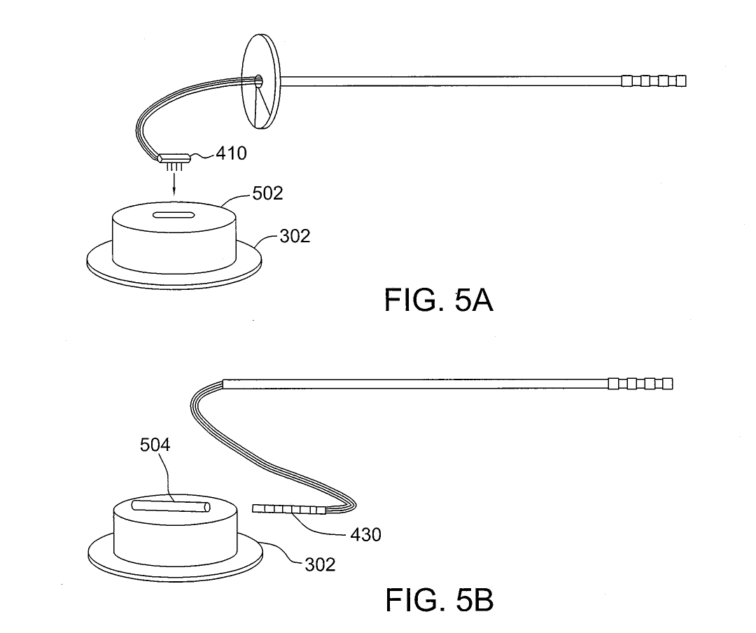

For example, FIG. 5A shows in-line connector 410 interfacing with an in-line connector receptacle 502 formed on the bottom of plug 302, and FIG. 5B shows cylindrical barrel connector 430 interfacing with a cylindrical barrel connector receptacle 504 formed on the bottom of plug 302. In-line connector receptacle 502 or cylindrical barrel connector receptacle 504 may be approximately 10 to 26 mm long and approximately 5 to 10 mm wide. Cylindrical barrel connector receptacle 504 includes contacts that align with cylindrical barrel connector 430 and couples to a complementary connector on plug 302. In-line connector receptacle 502 forms a socket that receives in-line contacts on in-line connector 410. In this embodiment, for isolation purposes, the in-line contacts have seals (not shown) around each conductive pin, around a perimeter of in-line connector 410, or around a perimeter of in-line connector receptacle 502. Similarly, cylindrical barrel connector receptacle 504 may incorporate O-ring seals that isolate each contact in cylindrical barrel connector 430.

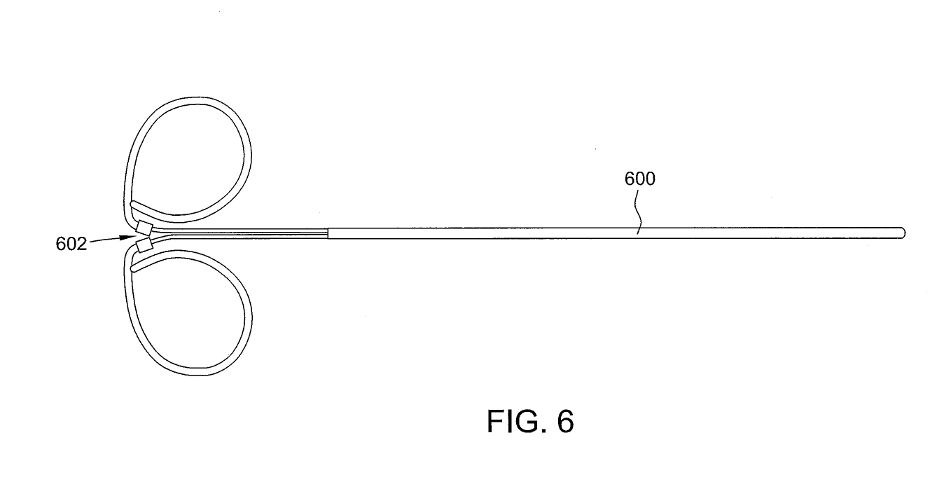

Apparatus 300 may be surgically implanted using a variety of techniques. For example, in some embodiments, cable conductors 306 and/or lead 304 are implanted using a splittable cannula 600 (shown in FIG. 6). Specifically, splittable cannula 600 is initially a tube that encircles cable conductors 306 and/or lead 304 to facilitate accurate positioning of cable conductors 306 and/or lead 304. Once cable conductors 306 and/or lead 304 are positioned as desired, splittable cannula 600 is split open (e.g., by spreading splittable cannula 600 along a longitudinal slit formed therein) to release cable conductors 306 and/or lead 304, and the split cannula 600 is removed and discarded. In FIG. 5, splittable cannula 600 is in the process of being split along a slit 602. In some embodiments, cable conductors 306 and/or lead 304 are positioned within a needle that facilitates placement of cable conductors 306 on dura 314 and/or insertion of lead 304 into brain 313.

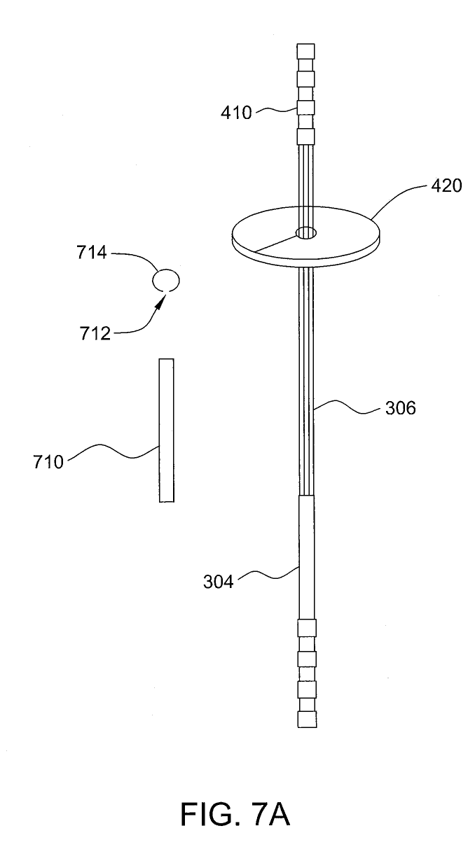

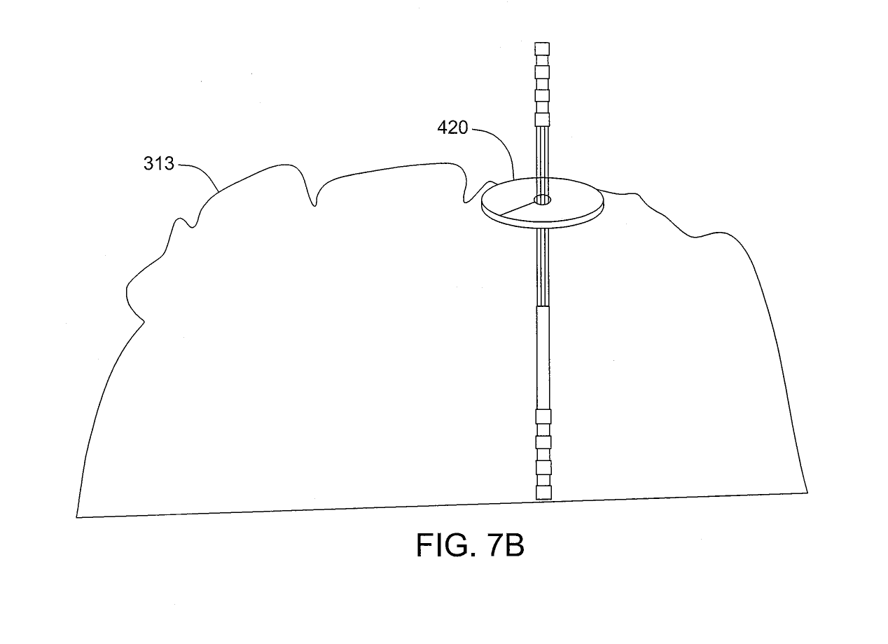

In some embodiments, apparatus 300 includes mechanisms to group cable conductors 306 together to prevent tissue ingrowth around cable conductors 306 if a distal end of lead 304 is made shorter. For example, as shown in FIGS. 7A and 7B, a distal end of lead 304 is made shorter such that a portion of cable conductors 306 are located inside brain 313. In this position, tissue may grow between cable conductors 306, increasing the difficulty of removing lead 304 if explant is required. Accordingly, to group cable conductors 306 together, a split segment of tubing 710 may be slipped over cable conductors 306. As shown in FIG. 7A, tubing 710 includes a slit 712 (seen best in a cross-section 714 of tubing 710) through which cable conductors 306 are slipped. Cross-section 714 is taken along a plane perpendicular to a longitudinal axis of tubing 710. Tubing 710 is cut to length such that a proximal end of tubing 710 is located at or near an outermost edge of the cortex, touching and/or passing through split washer 420.

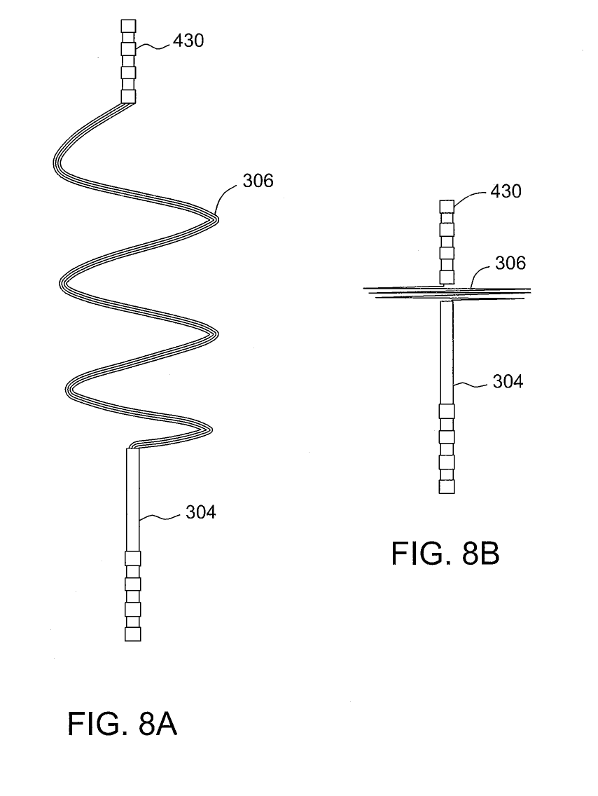

In some embodiments, to facilitate simplifying implantation of apparatus 300, cable conductors 306 are pre-shaped into a helix, as shown in FIGS. 8A and 8B. Specifically, cable conductors 306 may be pre-shaped by bending them past yield. As a result, and as shown in FIG. 8B, the cable conductors 306 collapse into a circular shape atop split washer 420 after connecting connector 410 or 430 to plug 302.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A neurostimulation apparatus for deep brain stimulation, the neurostimulation apparatus comprising:
a plug configured to be mounted to a burr hole formed in a skull of a subject;
a plurality of cable conductors configured to be coupled to the plug; and
a lead configured to be coupled to the plurality of cable conductors, the lead comprising at least one electrode configured to apply stimulation to the subject, wherein the plug is configured to generate and supply electrical stimulation pulses to the lead through the plurality of cable conductors.

2. The neurostimulation apparatus of claim 1, wherein each of the plurality of cable conductors has a diameter between approximately 0.0012 to 0.012 inches.

3. The neurostimulation apparatus of claim 1, wherein each of the plurality of cable conductors is coated in ethylene tetrafluoroethylene (ETFE).

4. The neurostimulation apparatus of claim 1, further comprising a split washer that substantially circumscribes the plurality of cable conductors, the split washer configured to rest on a dura of the subject.

5. The neurostimulation apparatus of claim 1, wherein the plurality of cable conductors are configured to be coupled to the plug using an in-line connector.

6. The neurostimulation apparatus of claim 1, wherein the plurality of cable conductors are configured to be coupled to the plug using a cylindrical barrel connector.

7. The neurostimulation apparatus of claim 1, wherein the plug comprises:
a battery configured to supply power for the plug;
a charging coil that facilitates inductively charging the plug;
processing circuitry configured to control operation of the plug; and
a communication antennae that facilitates communication with a device external to the neurostimulation apparatus.

8. A neurostimulation system comprising:
an apparatus for deep brain stimulation, the apparatus comprising:
a plug configured to be mounted to a burr hole formed in a skull of a subject;
a plurality of cable conductors coupled to the plug; and
a lead coupled to the plurality of cable conductors, the lead comprising at least one electrode configured to apply stimulation to the subject, wherein the plug is configured to generate and supply electrical stimulation pulses to the lead through the plurality of cable conductors; and
a controller device communicatively coupled to the apparatus and configured to control operation of the apparatus.

9. The neurostimulation system of claim 8, wherein each of the plurality of cable conductors has a diameter between approximately 0.0012 to 0.012 inches.

10. The neurostimulation system of claim 8, wherein each of the plurality of cable conductors is coated in ethylene tetrafluoroethylene (ETFE).

11. The neurostimulation system of claim 8, further comprising a split washer that substantially circumscribes the plurality of cable conductors, the split washer configured to rest on a dura of the subject.

12. The neurostimulation system of claim 8, wherein the plurality of cable conductors are configured to be coupled to the plug using an in-line connector.

13. The neurostimulation system of claim 8, wherein the plurality of cable conductors are configured to be coupled to the plug using a cylindrical barrel connector.

14. The neurostimulation system of claim 8, wherein the plug comprises:
a battery configured to supply power for the plug;
a charging coil that facilitates inductively charging the plug;
processing circuitry configured to control operation of the plug; and
a communication antennae that facilitates communication with the controller device.

15. A method for implanting a neurostimulation apparatus for deep brain stimulation, the method comprising:
inserting a lead into a brain of a subject, the lead including at least one electrode configured to apply stimulation to the subject;
coupling a plurality of cable conductors to the lead;
coupling the plurality of cable conductors to a plug; and
mounting the plug to a burr hole formed in a skull of the subject,
wherein the plug is configured to generate and supply electrical stimulation pulses to the lead through the plurality of cable conductors.

16. The method of claim 15, wherein coupling a plurality of cable conductors comprises coupling a plurality of cable conductors each having a diameter between approximately 0.0012 to 0.012 inches.

17. The method of claim 15, further comprising grouping the plurality of cable conductors together using a split segment of tubing.

18. The method of claim 15, further comprising implanting the plurality of cable conductors using a splittable cannula.

19. The method of claim 15, further comprising:
coupling a split washer to the plurality of cable conductors; and
positioning the split washer on a dura of the subject.

20. The method of claim 15, wherein coupling a plurality of cable conductors comprises coupling the plurality of cable conductors to the plug using at least one of an in-line connector and a cylindrical barrel connector.
